(19) United States
(12) Patent Application Publication
(10) Pub. No.: US 2013/0178720 A1
(43) Pub. Date: Jul. 11, 2013

(54) DEVICE, METHOD, AND PROGRAM FOR ESTIMATING BIORHYTHM

(71) Applicants: Naoya Sazuka, Tokyo (JP); Akane Sano, Tokyo (JP)

(72) Inventors: Naoya Sazuka, Tokyo (JP); Akane Sano, Tokyo (JP)

(73) Assignee: Sony Corporation, Tokyo (JP)

(21) Appl. No.: 13/810,317

(22) PCT Filed: May 26, 2011
(86) PCT No.: PCT/JP11/62095

§ 371 Date: Mar. 27, 2013

(30) Foreign Application Priority Data
Jul. 23, 2010 (JP) 2010-165786

(51) Int. Cl. A61B 5/0205; A61B 5/01; A61B 5/024

(52) U.S. Cl. CPC A61B 5/02055; A61B 5/024; A61B 5/01
USPC 600/301; 600/300; 600/500; 600/549

(57) ABSTRACT

Provided is a biorhythm estimation device including a reference rhythm curve storage unit configured to store a reference rhythm curve that is created as a biorhythm curve representing a property of intra-day fluctuation of an indicator value of biological information based on experimental subject samples, a biological information measurement unit configured to noninvasively measure the biological information of an experimental subject, an indicator value calculation unit configured to calculate the indicator value from a measurement value of the biological information, an experimental-subject rhythm curve creation unit configured to fit the reference rhythm curve to the indicator value of the experimental subject to create an experimental-subject rhythm curve whose target is the experimental subject, an experimental-subject rhythm curve storage unit configured to store the experimental-subject rhythm curve, and a biorhythm estimation unit configured to apply the indicator value to the experimental-subject rhythm curve to estimate biorhythm of the experimental subject.

### TECHNICAL FIELD

The present disclosure relates to a biorhythm estimation device, a biorhythm estimation method and a program.

### Background Art

Biorhythm is autonomous cyclic rhythm of an organism and adjusts changes in sleep, body temperature, blood pressure, autonomic nerves, or the like throughout the day. A deviation of biorhythm may have an influence on symptoms such as jet lag, sleep disorder, and seasonal depression, and treatment of biorhythm has attracted attention. Biorhythm may have an influence on exercise of physical ability or efficacy of a drug, and maximization of such effects has attracted attention. Accordingly, it is necessary to easily estimate biorhythm of an experimental subject.

For example, measuring a rectal temperature of an experimental subject for 24 hours or more and estimating a biorhythm curve is disclosed in the following Patent Literatures 1 and 2. Further, measuring a heart rate of an experimental subject for 24 hours or more and estimating a biorhythm curve is disclosed in the following Patent Literature 2.

### CITATION LIST

### Patent Literature

[Patent Literature 1] Japanese Patent Application Laid-open No. H6-189914A
[Patent Literature 2] Japanese Patent Application Laid-open No. H6-217946A

### SUMMARY OF INVENTION

### Technical Problem

However, an experimental subject should insert a measurement probe 10 cm or more into his or her anus in measurement of a rectal temperature and should attach a measurement patch to his or her chest in heart rate measurement. Further, continuously measuring biological information of an experimental subject over 24 hours or more is necessary in biorhythm estimation. Accordingly, the experimental subject experiences pain, discomfort or the like due to invasive measurement and suffers in daily life due to long-period measurement. Thus, in the related art, it is difficult to easily estimate the biorhythm of the experimental subject.

It is desirable to provide a biorhythm estimation device, a biorhythm estimation method, and a program capable of estimating biorhythm of an experimental subject based on noninvasively and easily measured biological information.

### Solution to Problem

According to an aspect of the present disclosure, there is provided a biorhythm estimation device including a reference rhythm curve storage unit configured to store a reference rhythm curve based on a plurality of experimental subject samples, the reference rhythm curve being created as a biorhythm curve representing a property of intra-day fluctuation of an indicator value of biological information, a biological information measurement unit configured to noninvasively measure the biological information of an experimental subject, an indicator value calculation unit configured to calculate the indicator value from a measurement value of the biological information, an experimental-subject rhythm curve creation unit configured to fit the reference rhythm curve to the indicator value calculated from the measurement value obtained by noninvasively measuring the biological information of the experimental subject to create an experimental-subject rhythm curve whose target is the experimental subject, an experimental-subject rhythm curve storage unit configured to store the experimental-subject rhythm curve, and a biorhythm estimation unit configured to apply the indicator value calculated from the measurement value obtained by noninvasively measuring the biological information of the experimental subject to the experimental-subject rhythm curve to estimate biorhythm of the experimental subject.

The reference rhythm curve may be created from the indicator value calculated from the measurement value of the biological information measured for the plurality of experimental subject samples for 24 hours or more, the experimental-subject rhythm curve creation unit may fit the reference rhythm curve to two or more indicator values calculated from the biological information noninvasively measured for the experimental subject twice or more at a time interval, and the biorhythm estimation unit may apply the two or more indicator values calculated from the biological information noninvasively measured for the experimental subject once or more to the experimental-subject rhythm curve.

Each of the reference rhythm curve and the experimental-subject rhythm curve may have a first rhythm curve representing a property of intra-day fluctuation of a first indicator value calculated from one or more pieces of biological information and a second rhythm curve representing a property of intra-day fluctuation of a second indicator value whose type is different from the first indicator value, the indicator value calculation unit calculates the first indicator value and the second indicator value from the one or more pieces of biological information noninvasively measured for the experimental subject once, and the biorhythm estimation unit may derive times t11 and t12 (t11<t12) corresponding the first indicator value on the first rhythm curve, derives times t21 and t22 (t21<t22) corresponding to the second indicator value on the second rhythm curve, and estimate the biorhythm of the experimental subject from a time corresponding to the smaller of a period |t11-t21| and a period |t12-t22|.

The first indicator value may be a pulse rate calculated from a pulse wave, and the second indicator value may be an AI value calculated from the pulse wave.

The first indicator value may be a pulse rate or an AI value calculated from a pulse wave, and the second indicator value may be an internal temperature of a mouth or an internal temperature of an ear.

The reference rhythm curve and the experimental-subject rhythm curve may have a rhythm curve representing a property of intra-day fluctuation of the indicator value calculated from the biological information, respectively, the indicator value calculation unit calculates a first indicator value and a second indicator value with different measurement time points from the biological information noninvasively measured for the experimental subject twice at a measurement interval within 12 hours, and the biorhythm estimation unit may derive one or more first times corresponding to the first indicator value on the experimental-subject rhythm curve, derives one or more second times corresponding to the second indicator value on the experimental-subject rhythm curve, and estimate the biorhythm of the experimental subject from the time corresponding to the closest period to the measurement interval among periods between the first times and the second times.

Each of the first and second indicator values may be a pulse rate or an AI value calculated from a pulse wave, or an internal temperature of a mouth or an internal temperature of an ear.

The reference rhythm curve and the experimental-subject rhythm curve may have a rhythm curve representing a property of intra-day fluctuation of the indicator value calculated from the biological information, respectively, the indicator value calculation unit calculates three or more indicator values with different measurement time points from the biological information noninvasively measured for the experimental subject three times or more at a time interval, and the biorhythm estimation unit may apply the three or more indicator values to the experimental-subject rhythm curve to estimate the biorhythm of the experimental subject from times corresponding to the three or more indicator values on the experimental-subject rhythm curve.

The indicator value may be a pulse rate or an AI value calculated from a pulse wave, or an internal temperature of a mouth or an internal temperature of an ear.

The reference rhythm curve may be prepared as a first rhythm curve whose target is a plurality of experimental subject samples having at least a first living-hour pattern, and a second rhythm curve whose target is a plurality of experimental subject samples having a second living-hour pattern different from the first living-hour pattern, and the experimental-subject rhythm curve creation unit may fit at least one of the first or second rhythm life that matches a living-hour pattern of the experimental subject to the indicator value to create the experimental-subject rhythm curve.

The biorhythm curve may be a single-mode function. The single-mode function may be a trigonometric function of a cycle of 24 hours.

Further, according to another aspect of the present disclosure, there is provided a biorhythm estimation method including preparing a reference rhythm curve based on a plurality of experimental subject samples as a biorhythm curve representing a property of intra-day fluctuation of an indicator value of the biological information, fitting the reference rhythm curve to the indicator value calculated from a measurement value obtained by noninvasively measuring the biological information of an experimental subject to create an experimental-subject rhythm curve whose target is the experimental subject, and applying the indicator value calculated from the measurement value obtained by noninvasively measuring the biological information of the experimental subject to the experimental-subject rhythm curve to estimate biorhythm of the experimental subject.

Further, according to another aspect of the present disclosure, there is provided a program for causing a computer to execute preparing a reference rhythm curve based on a plurality of experimental subject samples as a biorhythm curve representing a property of intra-day fluctuation of an indicator value of the biological information, fitting the reference rhythm curve to the indicator value calculated from a measurement value obtained by noninvasively measuring the biological information of an experimental subject to create an experimental-subject rhythm curve whose target is the experimental subject, and applying the indicator value calculated from the measurement value obtained by noninvasively measuring the biological information of the experimental subject to the experimental-subject rhythm curve to estimate biorhythm of the experimental subject.

### Advantageous Effects of Invention

As described above, according to the present disclosure, it is possible to provide a biorhythm estimation device, a biorhythm estimation method, and a program capable of estimating biorhythm of an experimental subject based on noninvasively and easily measured biological information.

### BRIEF DESCRIPTION OF DRAWINGS

FIG. 1 is a diagram illustrating an overview of a biorhythm estimation method according to an embodiment of the present disclosure.

FIG. 2 is a block diagram illustrating a configuration of a biorhythm estimation device according to an embodiment of the present disclosure.

FIG. 3 is a flow diagram illustrating an entire procedure of a biorhythm estimation method.

FIG. 4 is a diagram illustrating an AI value.

FIG. 5 is a flow diagram illustrating a procedure of creating a reference rhythm curve.

FIG. 6A is a diagram (1/4) illustrating the method of creating a reference rhythm curve.

FIG. 6B is a diagram (2/4) illustrating the method of creating a reference rhythm curve.

FIG. 6C is a diagram (3/4) illustrating the method of creating a reference rhythm curve.

FIG. 6D is a diagram (4/4) illustrating the method of creating a reference rhythm curve.

FIG. 7 is a flow diagram illustrating a biological information measurement procedure.

FIG. 8A is a diagram (1/3) illustrating a biological information measurement method.

FIG. 8B is a diagram (2/3) illustrating the biological information measurement method.

FIG. 8C is a diagram (3/3) illustrating the biological information measurement method.

FIG. 9 is a flow diagram illustrating a procedure of creating an experimental-subject rhythm curve.

FIG. 10 is a diagram illustrating a method of creating an experimental-subject rhythm curve.

FIG. 11 is a flow diagram illustrating a biorhythm estimation procedure based on a 1-point method.

FIG. 12 is a diagram illustrating a biorhythm estimation method based on a 1-point method.

FIG. 13 is a flow diagram illustrating a biorhythm estimation procedure based on a 2-point method.

FIG. 14A is a diagram (1/4) illustrating a biorhythm estimation method based on a 2-point method.

FIG. 14B is a diagram (2/4) illustrating the biorhythm estimation method based on a 2-point method.

FIG. 14C is a diagram (3/4) illustrating the biorhythm estimation method based on a 2-point method.

FIG. 14D is a diagram (4/4) illustrating the biorhythm estimation method based on a 2-point method.

FIG. 15 is a flow diagram illustrating a biorhythm estimation procedure based on a domain method.

FIG. 16 is a diagram illustrating a biorhythm estimation method based on the domain method.

### DESCRIPTION OF EMBODIMENTS

Hereinafter, preferred embodiments of the present disclosure will be described in detail with reference to the appended drawings. Note that, in this specification and the drawings, elements that have substantially the same function and structure are denoted with the same reference signs, and repeated explanation is omitted.

### 1. Overview of Biorhythm Estimation Method

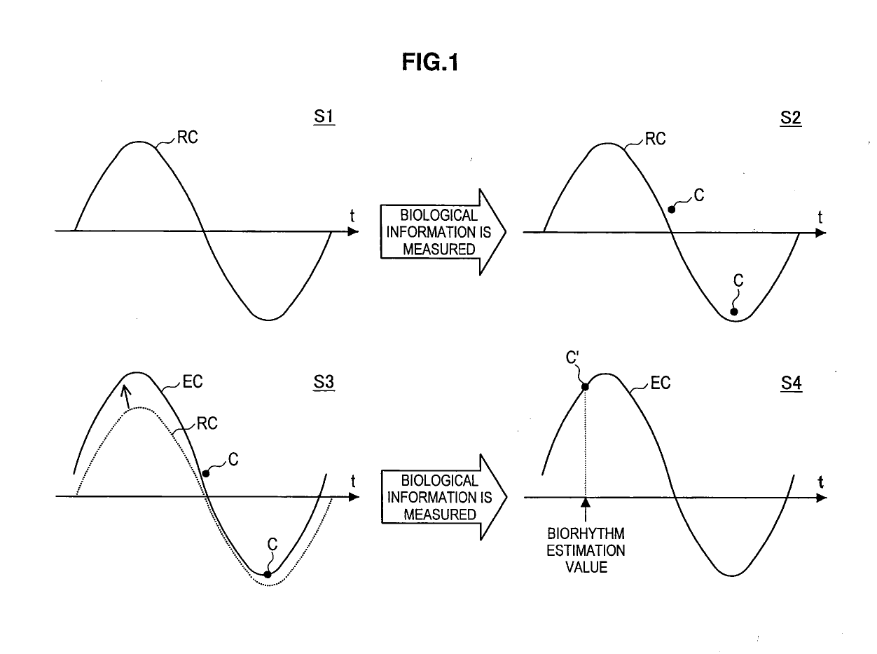

First, an overview of a biorhythm estimation method according to an embodiment of the present disclosure will be described with reference to FIG. 1. As illustrated in FIG. 1, in the biorhythm estimation method, a reference rhythm curve RC based on a plurality of experimental subject samples is first prepared as a biorhythm curve representing a property of intra-day fluctuation of an indicator value of biological information (step S1). Then, a reference rhythm curve RC is fitted to an indicator value C calculated from a measurement value obtained by noninvasively measuring biological information of the experimental subject to create an experimental-subject rhythm curve EC whose target is the experimental subject (steps S2 and S3). Also, an indicator value C′ calculated from the measurement value obtained by noninvasively measuring the biological information of the experimental subject is applied to the experimental-subject rhythm curve EC to estimate biorhythm of the experimental subject (step S4).

Here, the biological information which can be measured noninvasively and in a short time and from which the biorhythm is easily recognized, in other words, which has a goodness of fit of the biorhythm curve and the indicator value, is used in the estimation of the biorhythm. A pulse wave of an experimental subject or an experimental subject sample (hereinafter also referred to as an experimental subject or the like), an internal temperature of a mouth or an ear, or the like is used as the biological information, but the biological information is not limited thereto.

Accordingly, the experimental-subject rhythm curve EC can be created from measurement values measured twice or more at a time interval without continuously measuring the biological information for 24 hours, and the biorhythm can be estimated from measurement values of one time or more. The reference rhythm curve RC and the experimental-subject rhythm curve EC are created, for example, as a single-mode function such as a trigonometric function of a cycle of 24 hours.

Accordingly, the experimental subject does not experience pain, discomfort or the like due to invasive measurement and does not suffer in daily life due to long-period measurement, unlike a biorhythm estimation method of the related art. Thus, it is possible to estimate the biorhythm of the experimental subject based on the noninvasively and easily measured biological information.

### 2. Configuration of Biorhythm Estimation Device 1

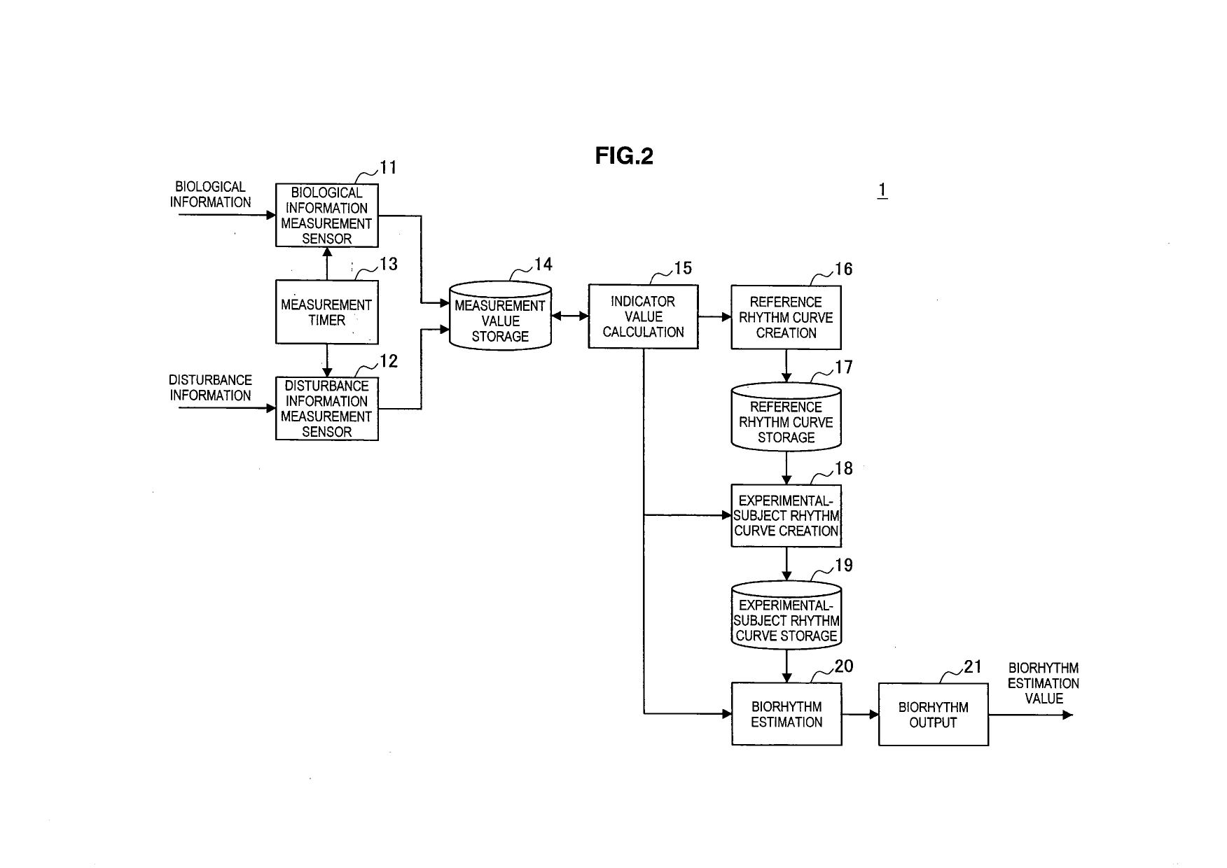

Next, a primary functional configuration of a biorhythm estimation device 1 according to an embodiment of the present disclosure will be described with reference to FIG. 2. As illustrated in FIG. 2, the biorhythm estimation device 1 includes a biological information measurement sensor 11, a disturbance information measurement sensor 12, a measurement timer 13, a measurement value storage unit 14, an indicator value calculation unit 15, a reference rhythm curve creation unit 16, a reference rhythm curve storage unit 17, an experimental-subject rhythm curve creation unit 18, an experimental-subject rhythm curve storage unit 19, a biorhythm estimation unit 20, and a biorhythm output unit 21.

In the biorhythm estimation device 1 illustrated in FIG. 2, creation of a reference rhythm curve RC, creation of an experimental-subject rhythm curve EC, and estimation of biorhythm are performed. Alternatively, the biorhythm estimation device 1 may be configured to store a reference rhythm curve RC created by an external device different from the biorhythm estimation device 1. In this case, the reference rhythm curve creation unit 16 can be omitted.

The biological information measurement sensor 11 is a sensor for measuring biological information such as a pulse wave of an experimental subject or the like, an internal temperature of a mouth or an ear, or the like. The pulse wave is measured as a photoelectric pulse wave or a pressure pulse wave from a fingertip or the like of the experimental subject or the like, and the internal temperature of the mouth or the ear is measured as a body temperature of the experimental subject or the like. The measurement value of the biological information is stored in the measurement value storage unit 14 for a subsequent process.

The disturbance information measurement sensor 12 is a sensor for measuring disturbance information generated at the time of measurement of the biological information due to a motion or the like of the experimental subject or the like (e.g., an acceleration due to an action of the experimental subject or the like). The measurement value of the disturbance information is also stored in the measurement value storage unit 14 for a subsequent process.

The measurement timer 13 supplies timing information indicating a predetermined measurement timing to the biological information measurement sensor 11 and the disturbance information measurement sensor 12.

The indicator value calculation unit 15 calculates an indicator value from the biological information based on the measurement values of the biological information of the experimental subject or the like and the disturbance information. The indicator value is an average pulse rate calculated from the pulse wave, an average AI (Augmentation Index) value, an average internal temperature of the mouth or the ear, or the like, but is not limited thereto.

The measurement values of the biological information and the disturbance information are read from the measurement value storage unit 14 in order to calculate the indicator value. The indicator value of the experimental subject sample is supplied to the reference rhythm curve creation unit 16. Further, the indicator value of the experimental subject is supplied to the experimental-subject rhythm curve creation unit 18 and used for creation of an experimental-subject rhythm curve EC and/or supplied to the biorhythm estimation unit 20 and used for estimation of the biorhythm.

The reference rhythm curve creation unit 16 creates the reference rhythm curve RC from the indicator value that is calculated from the measurement value of the biological information measured for 24 hours or more for a plurality of experimental subject samples. The reference rhythm curve RC is a curve representing an average property of intra-day fluctuation of the indicator value whose target is a plurality of experimental subject samples. The created reference rhythm curve RC is stored in the reference rhythm curve storage unit 17.

The experimental-subject rhythm curve creation unit 18 fits the reference rhythm curve RC to two or more indicator values C calculated from the biological information noninvasively measured for the experimental subject twice or more at a time interval to create the experimental-subject rhythm curve EC. The experimental-subject rhythm curve EC is a curve representing a property of intra-day fluctuation of the indicator value whose target is the experimental subject. The indicator value C from the indicator value calculation unit 15 and the reference rhythm curve RC from the reference rhythm curve storage unit 17 are supplied to the experimental-subject rhythm curve creation unit 18. The created experimental-subject rhythm curve EC is stored in the experimental-subject rhythm curve storage unit 19.

The biorhythm estimation unit 20 applies two or more indicator values C′ calculated from the biological information noninvasively measured one time or more to the experimental-subject rhythm curve EC to estimate the biorhythm of the experimental subject. The indicator values C′ of the experimental subject from the indicator value calculation unit 15 and the experimental-subject rhythm curve EC from the experimental-subject rhythm curve storage unit 19 are supplied to the biorhythm estimation unit 20. The estimation value of the biorhythm is supplied to the biorhythm output unit 21.

The biorhythm output unit 21 outputs the biorhythm estimation value. The biorhythm estimation value is supplied or output to the inside of the biorhythm estimation device 1 or to an external display device, an external printing device or an external storage device (all not shown). As the biorhythm, a time corresponding to the measurement value on the experimental-subject rhythm curve EC may be output or a difference (a biorhythm deviation) between a time corresponding to the measurement value on the experimental-subject rhythm curve EC and an actual measurement time may be output.

Here, in the above functional configuration, the indicator value calculation unit 15, the reference rhythm curve creation unit 16, the experimental-subject rhythm curve creation unit 18, the biorhythm estimation unit 20, and the biorhythm output unit 21 are configured of an arithmetic processing device such as a CPU or a DSP (digital signal processing device). The measurement value storage unit 14, the reference rhythm curve storage unit 17, and the experimental-subject rhythm curve storage unit 19 are configured of an internal storage device such as a flash memory, a hard disk drive, or an external storage device such as Blu-ray disc drive. Also, the CPU develops and executes, on a RAM, a program read from a ROM or the like to realize a biorhythm estimation method. Further, at least a part of the above functional configuration may be configured as hardware such as a dedicated logic.

### 3. Operation of Biorhythm Estimation Device 1

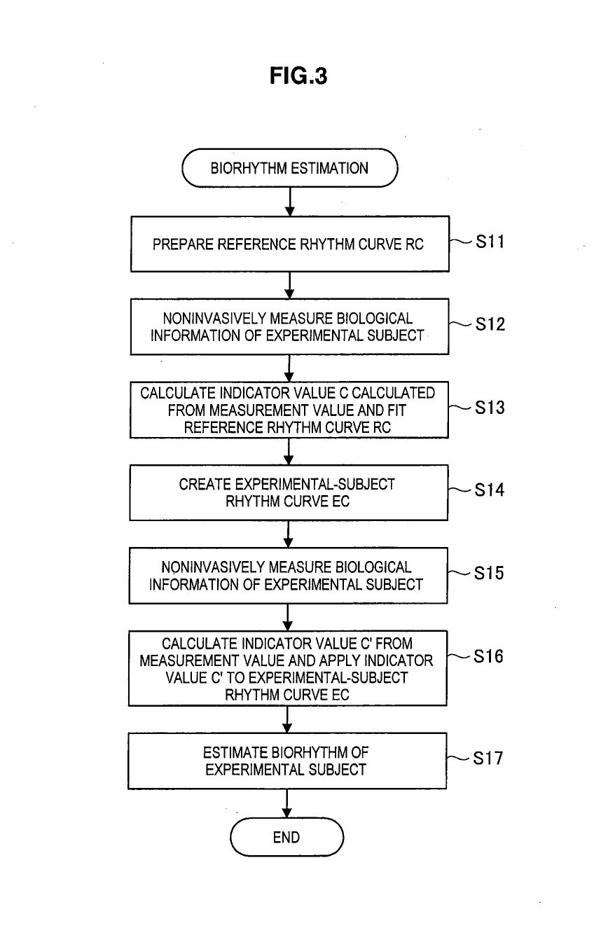

Next, operation of the biorhythm estimation device 1 according to the embodiment of the present disclosure will be described with reference to FIGS. 3 to 16.

As illustrated in FIG. 3, first, a reference rhythm curve RC is prepared (step S11). The reference rhythm curve RC is a biorhythm curve representing a property of intra-day fluctuation of an indicator value of biological information using a single-mode function such as a trigonometric function of a cycle of 24 hours, and is a biorhythm curve representing an average property of intra-day fluctuation of indicator values whose target is a plurality of experimental subject samples.

Then, the biological information of the experimental subject is noninvasively measured (step S12), and the reference rhythm curve RC is fitted to an indicator value C calculated from the measurement value (step S13) to create an experimental-subject rhythm curve EC (step S14). The experimental-subject rhythm curve EC is a biorhythm curve representing a property of intra-day fluctuation of the indicator value whose target is the experimental subject.

Then, the biological information of the experimental subject is noninvasively measured (step S15) and an indicator value C′ calculated from a measurement value is applied to the experimental-subject rhythm curve EC (step S16) to estimate the biorhythm of the experimental subject (step S17). Further, some of the indicator values C of step S13 may be used as the indicator value C′ of step S16.

Here, biological information which can be measured noninvasively and in a short time and from which the biorhythm is easily recognized, in other words, which has a goodness of fit of the biorhythm curve and the indicator value calculated from the measurement value, is used in the estimation of the biorhythm. Such biological information includes a photoelectric pulse wave or a pressure pulse wave of a fingertip or the like of the experimental subject or the like, an internal temperature of a mouth or an ear, or the like, but is not limited thereto.

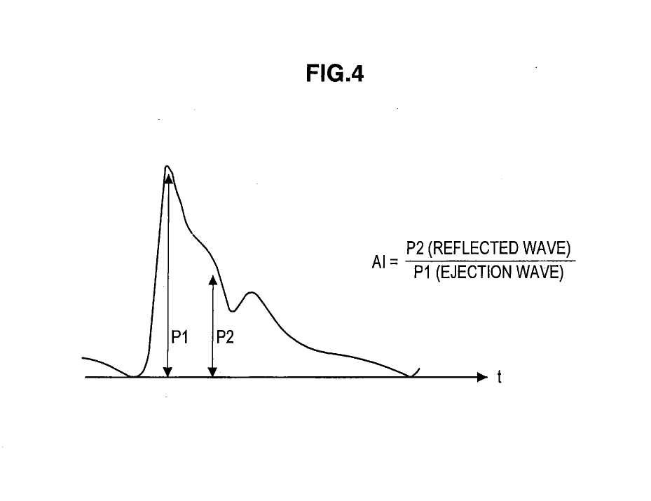

Hereinafter, a case in which a pulse wave is measured as the biological information, and a pulse rate and an AI value are calculated as indicator values from a measurement value of the pulse wave will be described. Here, the pulse wave is a pressure wave generated in a vessel when blood is sent from a heart. As illustrated in FIG. 4, the pulse wave includes a pressure wave generated due to contraction of the heart (ejection wave), and a pressure wave generated due to reflection of the ejection wave in a bifurcation of an artery or a peripheral blood vessel (a reflected wave). Also, the AI value is a ratio P2/P1 of a peak P2 of the reflected wave and a peak P1 of the ejection wave, and is used as an indicator indicating a load on the heart or hardness of an artery.

### [3-1. Preparation of Reference Rhythm Curve RC]

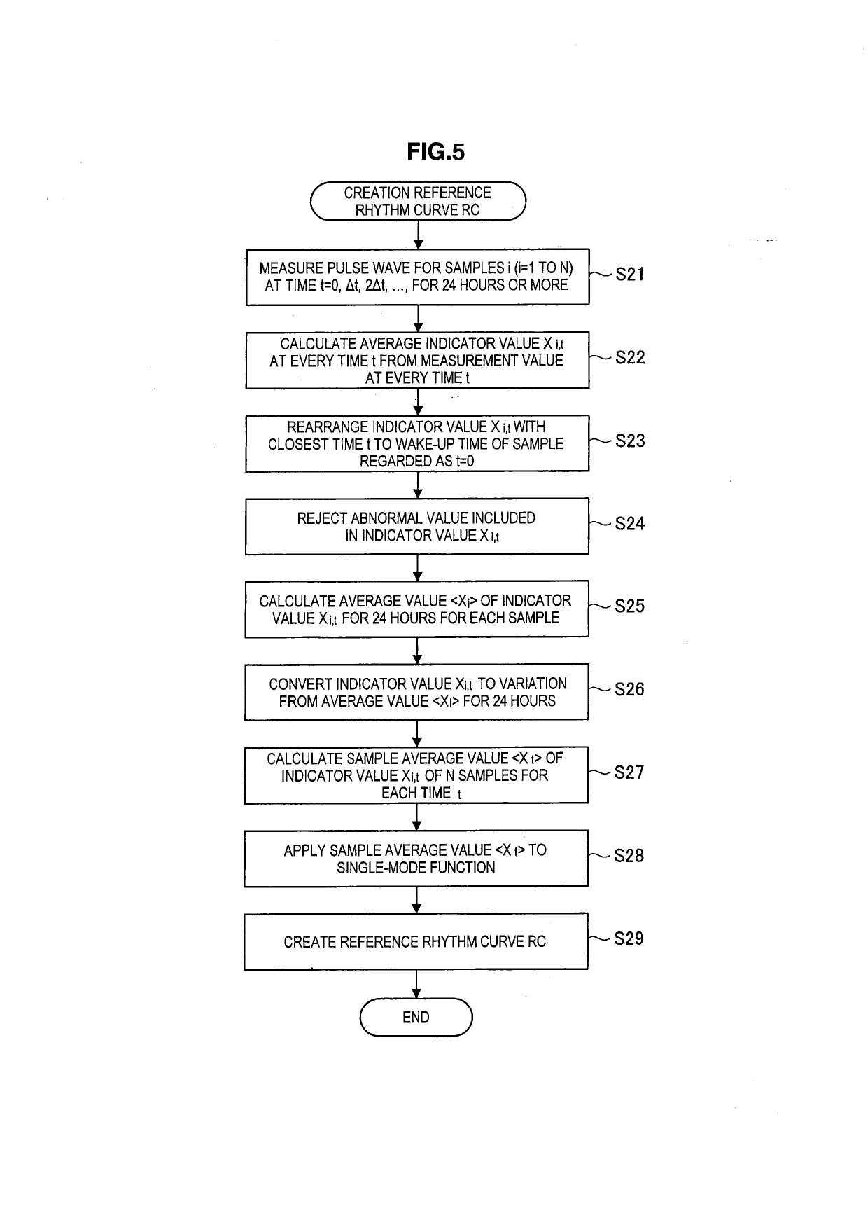

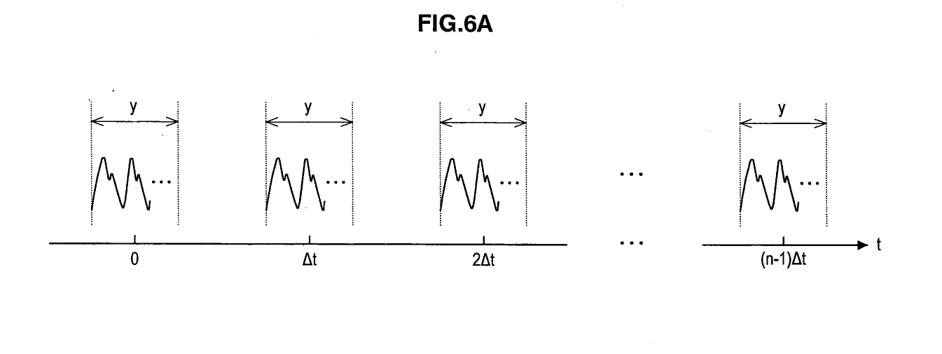

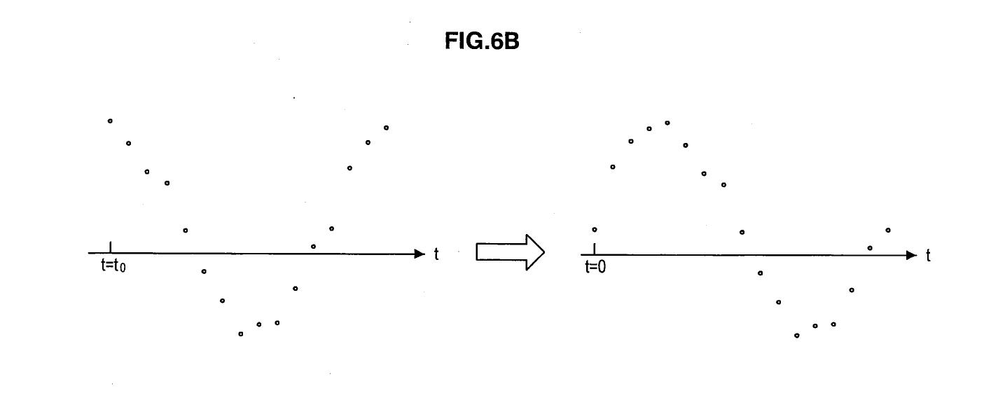

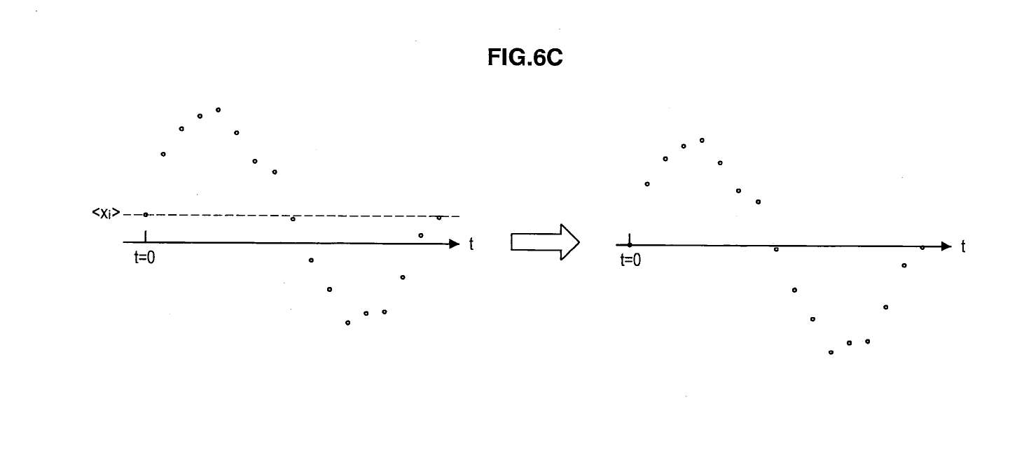

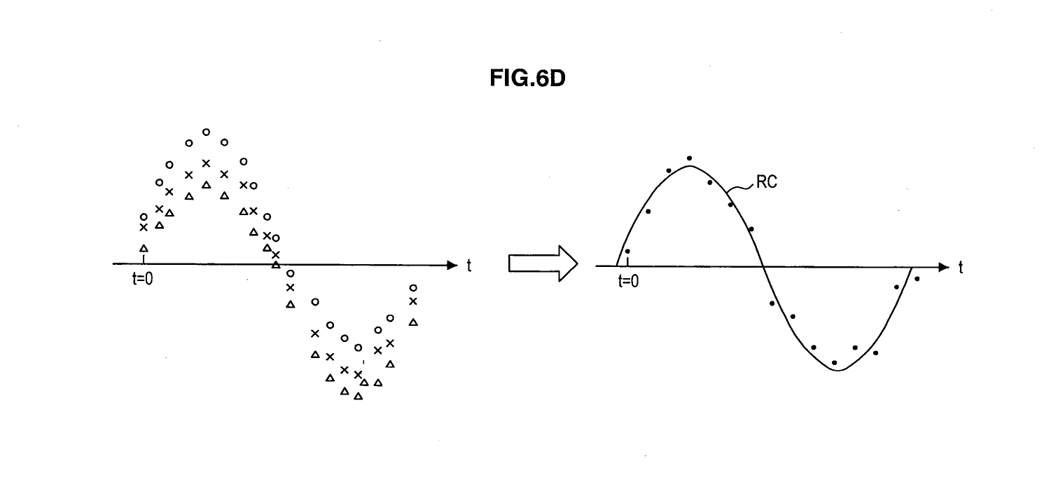

A method of creating the reference rhythm curve RC is illustrated in FIGS. 5 and 6A to 6D. The reference rhythm curve RC is created by measuring biological information for a plurality of experimental subject samples for 24 hours or more and calculating an indicator value from measurement values for 24 hours or more. Hereinafter, a procedure of creating the reference rhythm curve RC will be described with reference to FIG. 5.

In the reference rhythm curve creation unit 16, the reference rhythm curve RC is created from the indicator value calculated from the measurement value of the biological information measurement sensor 11, and the created reference rhythm curve RC is stored in the reference rhythm curve storage unit 17. In the creation of the reference rhythm curve RC, the measurement value of the biological information may be measured as a continuous measurement value, but a case in which the measurement value of the biological information is measured as a discontinuous measurement value will be described hereinafter.

First, a pulse wave for each of experimental subject samples of N persons (i=1 to N) (hereinafter referred to as samples) is measured for 24 hours or more (step S21). As illustrated in FIG. 6A, the pulse wave is measured in a measurement period of time y at every time t (=0, Δt, 2Δt, (n−1)Δt) in a measurement period Δt. Further, n corresponding to the number of measurements of each sample i is any value for each sample. Here, the measurement period Δt is 0.5 hours, 1 hour, or the like, and the measurement period of time y ranges from tens of seconds to several minutes.

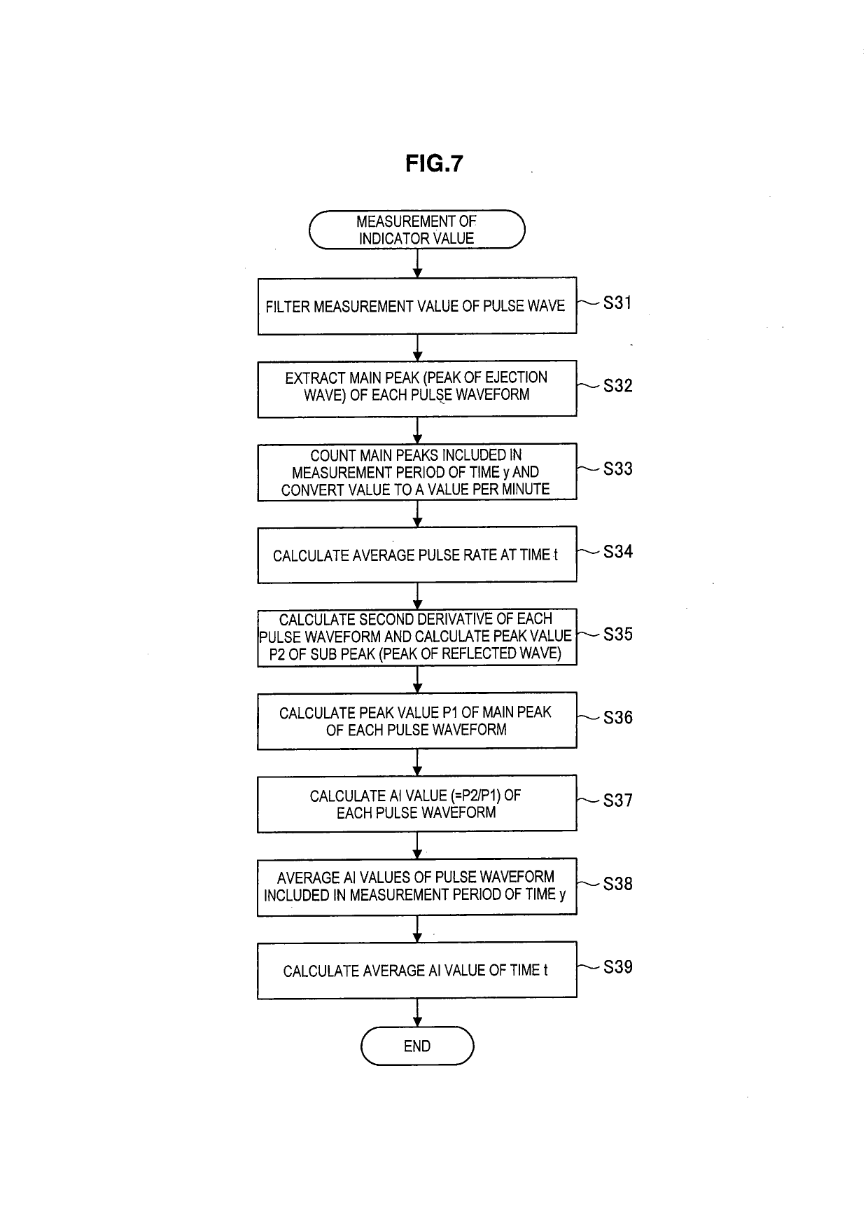

Then, an average indicator value X_{i,t} (e.g., an average pulse rate or an average AI value) is calculated from the pulse wave of the measurement period of time y at every time t for each sample i (step S22). In the indicator value calculation unit 15, an indicator value X_{i,t} at every time t is calculated from the measurement values of the biological information and the disturbance information. Further, a procedure of calculating the indicator value X_{i,t} will be described with reference to FIG. 7.

In the calculation of the indicator value X_{i,t}, as illustrated in FIG. 7, first, the measurement value of the pulse wave in the measurement period of time y (a series of pulse waveforms) is filtered (step S31). The measurement value of the pulse wave is shaped using a low pass filter or a band pass filter.

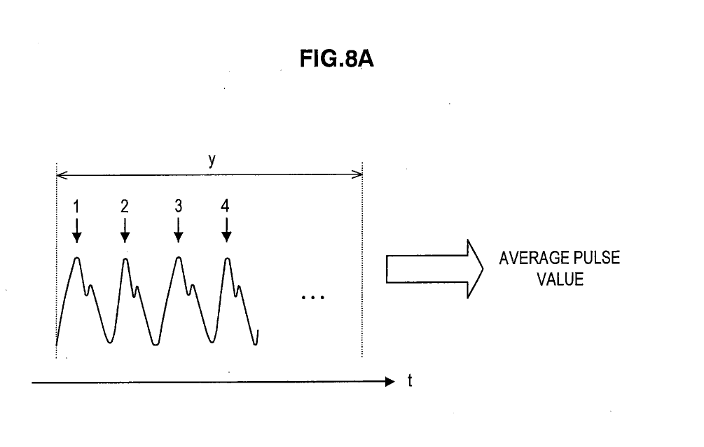

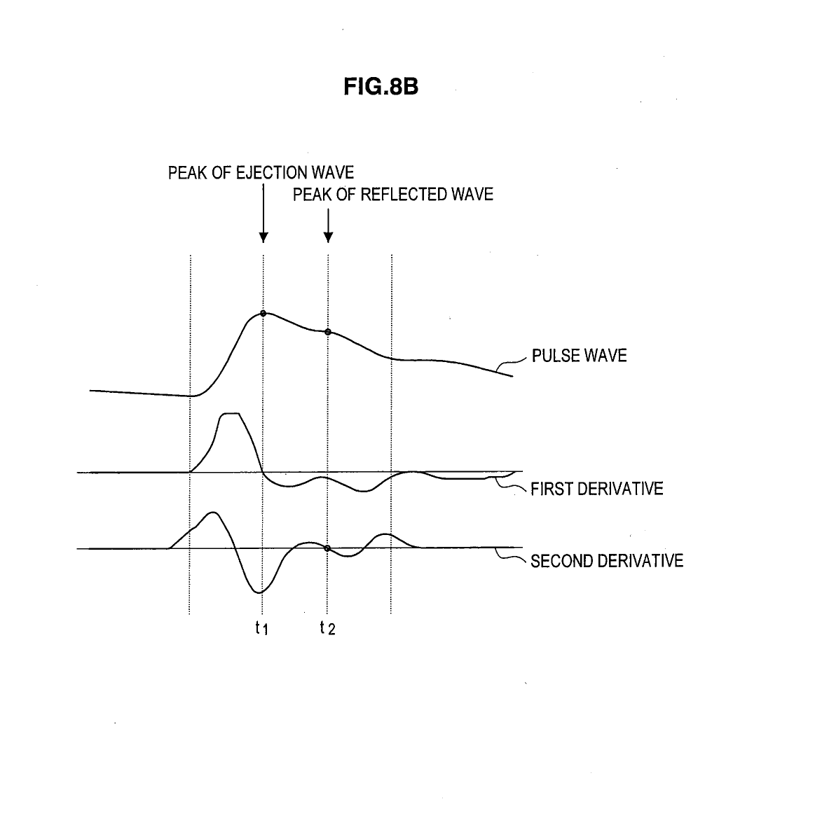

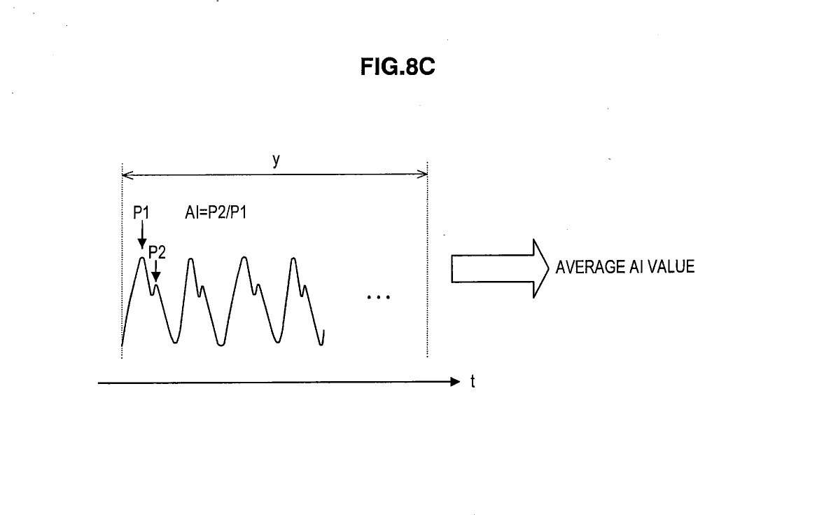

Next, as illustrated in FIG. 8A, a main peak (a peak of the ejection wave) of each pulse waveform (each pulse waveform constituting a series of pulse waveforms) is extracted (step S32). Also, the main peaks included in the measurement period of time y are counted (corresponding to count values “1, 2, 3, . . . ” in FIG. 8A) and converted to a value per minute (step S33) to create an average pulse rate at a time t as the indicator value X_{i,t} (step S34).

Next, as illustrated in FIG. 8B, a second derivative of each pulse waveform is calculated and a time t₂ of a change point at which a second derivative value is changed at a second time from a positive value to a negative value is calculated. Further, the calculation of the second derivative of each pulse waveform is performed, for example, using a difference value between adjacent sample values measured in a sampling cycle of about 200 Hz or a difference value weighted by an adjacent sample value.

Next, as illustrated in FIG. 8C, a peak value P1 corresponding to the main peak and a peak value P2 of a sub-peak corresponding to a change point (a peak of the reflected wave) are calculated for each pulse waveform (steps S35 and S36). The peak value P2/the peak value P1 is then calculated as the AI value for each pulse waveform (step S37). Also, the AI values of the pulse waveform included in the measurement period of time y are averaged (step S38) to calculate an average AI value of a time t as the indicator value X_{i,t} (step S39).

After the indicator value X_{i,t} is calculated, the process returns to the procedure of creating the reference rhythm curve RC illustrated in FIG. 5. The following procedure is performed for each indicator (e.g., an average pulse rate or an average AI value). As illustrated in FIG. 6B, the indicator value X_{i,t} is then rearranged with the closest time t to a wake-up time of each sample regarded as t=0 (step S23). A wake-up time of each sample may be measured by the disturbance information measurement sensor 12 attached to the sample and recorded by a person performing measurement.

Then, an abnormal value included in the indicator value X_{i,t} is rejected (step S24). For example, if a difference value between indicator values in tandem (e.g., indicator values X_{i,t} and X_{i,t+Δt}) or indicator values within a predetermined time (e.g., indicator values X_{i,t}, X_{i,t+Δt} and X_{i,t+2Δt}) exceeds a predetermined threshold value, an unstable state of the sample is confirmed from the measurement value of the biological information or the disturbance information, and the indicator value is rejected as an abnormal value. Further, when the number of abnormal values exceeds a predetermined threshold value, the experimental subject or the like is requested to perform re-measurement of the pulse wave via a notification unit (not shown) or the like.

Next, as illustrated in FIG. 6C, an average value <Xᵢ> of the indicator value X_{i,t} for 24 hours is calculated for each sample (step S25), and the indicator value X_{i,t} is converted to a variation from the average value <Xᵢ> for 24 hours (X_{i,t}→X_{i,t}-<Xᵢ>) (step S26).

Next, as illustrated in FIG. 6D, a sample average value <Xᵢ> of the indicator value X_{i,t} of the N persons is calculated for each time t (<Xᵢ>=avr(X_{i,t}) (step S27). Also, the sample average value <Xₜ> is applied to a single-mode function such as a trigonometric function of a cycle of 24 hours using a least-square method or the like (step S28) to create the reference rhythm curve RC (step S29). Here, the trigonometric function of a cycle of 24 hours is represented, for example, as Equation (1). In the application of the sample average value <Xₜ>, unknown numbers Aₜₘₚ and Bₜₘₚ are estimated.

RC(t)=Aₜₘₚsin(wt+Bₜₘₚ), w=2π/24 hours (1)

In the measurement of the pulse wave, each experimental subject sample may go to bed or wake up according to his or her living-hour pattern or may go to bed or wake up at a time defined as a measurement condition. Here, a plurality of experimental subject samples and experimental subjects are considered as belonging to various living-hour pattern types (e.g., a morning type, an evening type, and an intermediate type). Accordingly, the living-hour patterns of the experimental subject samples are specified and then one or more reference rhythm curves (e.g., a morning type rhythm curve or an evening type rhythm curve) corresponding to the various living-hour patterns may be created.

Further, a difference between biorhythm curves according to the living-hour patterns is described in, for example, “E. K. Baehr et al., Individual differences in the phase and amplitude of the human circadian temperature rhythm: with an emphasis on morningness-eveningness, J. Sleep Res, 2000, 9, 117-127.” Further, when the biorhythm curve according to the living-hour pattern is created, the living-hour pattern of an experimental subject or the like may be identified using, for example, morning-type and evening-type questionnaire (“Morningness Eveningness Questionnaire, Home and Ostberg 1976.).”

### [3-2. Creation of Experimental-Subject Rhythm Curve EC]

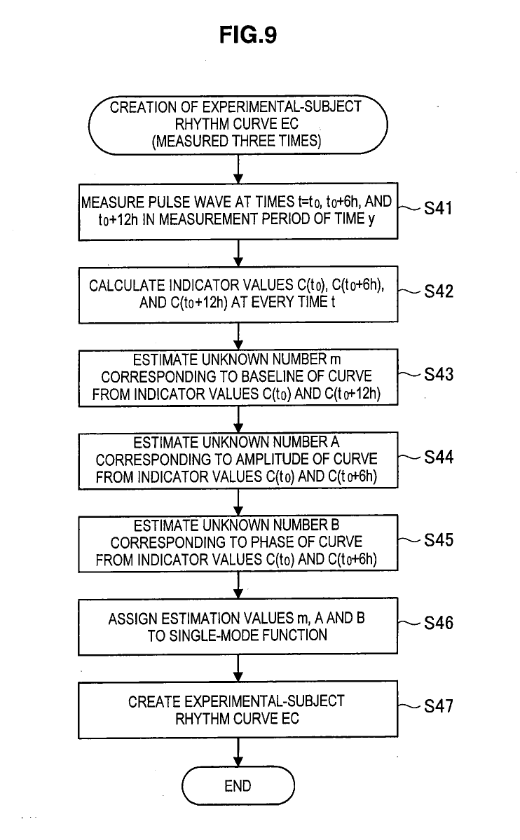

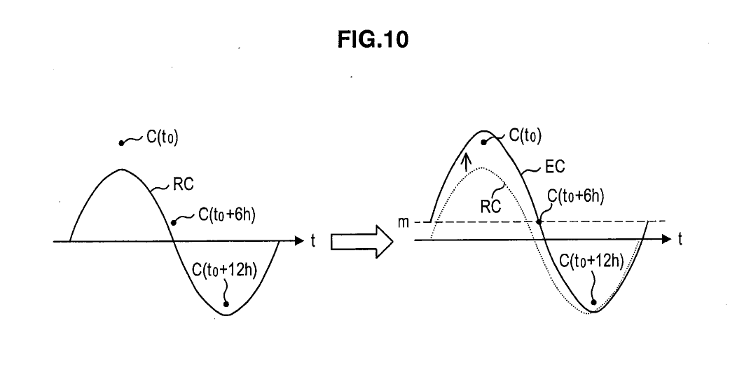

A method of creating the experimental-subject rhythm curve EC is illustrated in FIGS. 9 and 10. The experimental-subject rhythm curve EC is created by noninvasively measuring the biological information of the experimental subject twice or more at a time interval, calculating two or more indicator values C from measurement values measured twice or more, and fitting the reference rhythm curve RC to the two or more indicator values C. Further, each of the measurement values measured twice or more corresponds to each of the two or more indicator values C. Here, when the living-hour pattern of the experimental subject is considered, the experimental-subject rhythm curve EC is created by fitting the reference rhythm curve RC matching the living-hour pattern of the experimental subject to the two or more indicator values C.

In the experimental-subject rhythm curve creation unit 18, the reference rhythm curve RC is fitted to the indicator value C calculated from the measurement value of the biological information measurement sensor 11 to create the experimental-subject rhythm curve EC, and the created experimental-subject rhythm curve EC is stored in the experimental-subject rhythm curve storage unit 19. In the creation of the experimental-subject rhythm curve EC, the measurement value of the biological information is measured as a discontinuous measurement value. Further, the experimental-subject rhythm curve EC is created for each indicator (e.g., an average pulse rate or an average AI value).

The experimental-subject rhythm curve EC is created using measurement values of two times or measurement values of three times. Hereinafter, a case in which measurement is performed three times at any time t₀, after 6 hours from the time t₀, and after 12 hours from the time t₀ will be mainly described.

First, a case in which measurement values of three times are used will be described with reference to FIG. 9. As illustrated in FIG. 9, first, a pulse wave is measured three times, for example, at any time t₀, t₀+6 h, and t₀+12 h (step S41). For example, if a time t is 9 o'clock, 15 o'clock, and 21 o'clock, a value close to a maximum value in a day or a minimum value in a day of a pulse rate or an AI value can be measured and a highly accurate experimental-subject rhythm curve EC can be created. Next, the indicator values C(t₀), C(t₀+6 h), and C(t₀+12 h) are calculated at every time t in the procedure described with reference to FIG. 7 (step S42).

The indicator values C(t₀), C(t₀+6 h) and C(t₀+12 h) corresponding to the measurement values of three times can be applied to a single-mode function similar to Equation (1), which represents the reference rhythm curve RC, as shown in Equation (2). Here, an unknown number m corresponding to a difference between a change center of the indicator value and a baseline (amplitude center) of the trigonometric function, an unknown number A corresponding to the amplitude of the trigonometric function, and an unknown number B corresponding to a phase of the trigonometric function are estimated in the above application.

Here, the experimental-subject rhythm curve EC is created by adjusting the baseline using the unknown number m, the amplitude using the unknown number A, and the phase using the unknown number B with respect to the reference rhythm curve RC.

EC(t)=m+A sin(Z), Z=wt+B, w=2π/24 hours (2)

The indicator values C(t₀), C(t₀+6 h) and C(t₀+12 h) are represented as Equations (3), (4) and (5).

C(t₀)=m+A sin(Z) (3)

C(t₀+6 h)=m+A cos(Z) (4)

C(t₀+12 h)=m−A sin(Z) (5)

The unknown number m is then estimated as Equation (6) using the indicator values C(t₀) and C(t₀+12 h) (step S43). Similarly, the unknown number A is estimated as Equation (7) using the indicator values C(t₀) and C(t₀+6 h) or the indicator values C(t₀+6 h) and C(t₀+12 h) (step S44). Further, since the value w and the value t are known, the unknown number B is estimated using the indicator values C(t₀) and C(t₀+6 h) or the indicator values C(t₀+6 h) and C(t₀+12 h) (step S45). Also, the estimation values m, A and B are applied to Equation (2) (step S46) to create the experimental-subject rhythm curve EC (step S47).

m=(C(t₀)+C(t₀+12 h))/2 (6)

A=√((C(t₀)−m)²+(C(t₀+6 h)−m)²) (7)

Next, a case in which measurement is performed three times at any time t₀, after 3 hours from the time t₀, and after 6 hours from the time t₀ will be briefly described. In this case, indicator values C(t₀), C(t₀+3 h), and C(t₀+6 h) are represented as Equations (8), (9) and (10).

C(t₀)=m+A sin(Z) (8)

C(t₀+3 h)=m+A(sin Z+cos Z)/√2 (9)

C(t₀+6 h)=m+A cos Z (10)

where Z=wt₀+B.

The unknown numbers m and A are estimated as Equations (11) and (12) from Equations (8) to (10).

m=C{C(t₀)+C(t₀+6 h)−√2·C(t₀+3h)}/(2−√2) (11)

A=·{(C(t₀)−m)²+(C(t₀+6 h)−m)²} (12)

An unknown number A is estimated by assigning Equation (11) to Equation (12).

An unknown number Z is estimated by assigning estimation values m and A to Equations (8) and (10). Here, since the value w and the value t are known, the unknown number B is obtained. Also, as the estimation values m, A and B are assigned to Equation (2), the experimental-subject rhythm curve EC is created.

Next, a case in which measurement is performed two times at any times t₁ and t₂ will be briefly described. In this case, indicator values C(t₁) and C(t₂) are represented as Equations (13) and (14).

C(t₁)=m+A sin(Z+t₁) (13)

C(t₂)=m+A sin(Z+t₂) (14)

where Z=wt+B.

Unknown numbers m and A are estimated as Equations (15) and (16) by solving a simultaneous equation of Equations (13) and (14).

m={C(t₁)sin(Z+t₂)−C(t₂)sin(Z+t₁)}/{sin(Z+t₂)−sin(Z+t₁)} (15)

A={C(t₂)−C(t₁)}/{sin(Z+t₂)−sin(Z+t₁)} (16)

Further, an estimation value Bₜₘₚ used for creation of the reference rhythm curve RC is applied as the unknown number B. Since the estimation value Bₜₘₚ is estimated from an average value <Xt> of N samples and has higher accuracy than the unknown number B, the estimation value is treated as a constant number. Also, the estimation values m, A and B (=Bₜₘₚ) are assigned to Equation (2) to create the experimental-subject rhythm curve EC.

### [3-3. Biorhythm Estimation]

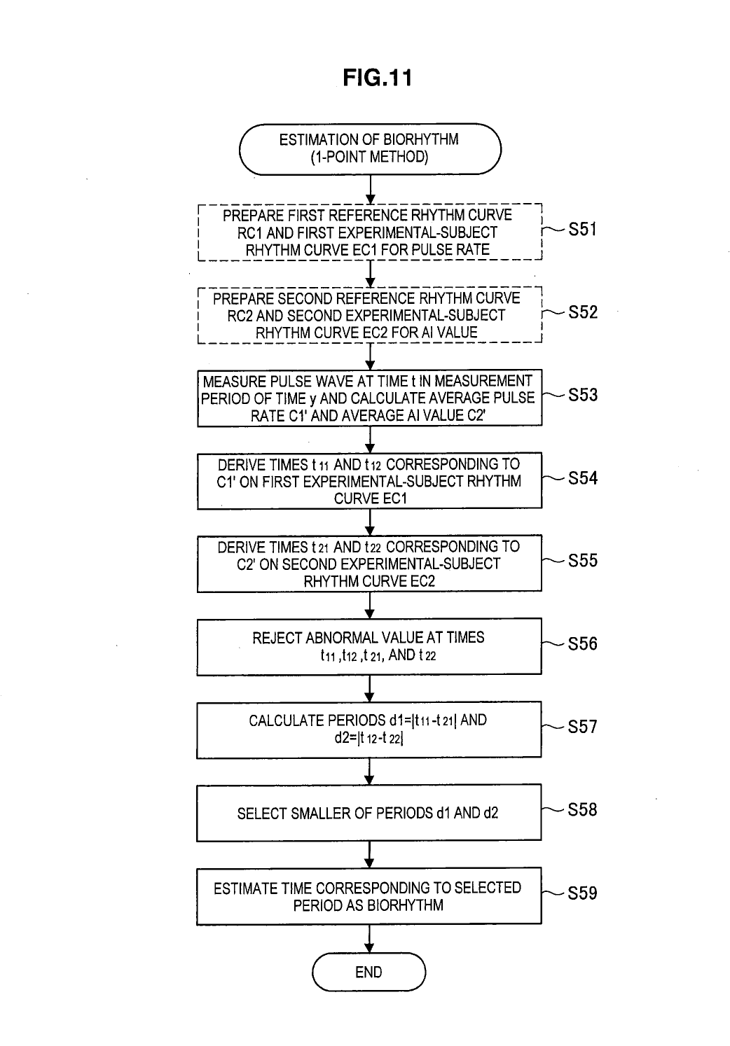

A method of estimating biorhythm of an experimental subject is shown in FIGS. 11 to 16. Hereinafter, biorhythm estimation procedures based on a 1-point method, a 2-point method and a domain method will be described in order.

In the biorhythm estimation unit 20, two or more indicator values C′ calculated from the biological information measured noninvasively one time or more for an experimental subject are applied to the experimental-subject rhythm curve EC to estimate the biorhythm of the experimental subject, and the biorhythm estimation value is supplied to the biorhythm output unit 21. Here, when the living-hour pattern of the experimental subject is considered, the biorhythm of the experimental subject is estimated by applying two or more indicator values C′ calculated from the measurement value of the biological information of one time or more to the experimental-subject rhythm curve EC in which the living-hour pattern of the experimental subject is considered.

(Biorhythm Estimation Based on 1-point Method)

In the 1-point method, first and second reference rhythm curves RC1 and RC2 and first and second experimental-subject rhythm curves EC1 and EC2 are used. The first reference rhythm curve RC1 and the first experimental-subject rhythm curve EC1 are curves representing a property of intra-day fluctuation of the first indicator value C1′. The second reference rhythm curve RC2 and the second experimental-subject rhythm curve EC2 are curves representing a property of intra-day fluctuation of a second indicator value C2′ different from the first indicator value C1′.

In the indicator value calculation unit 15, the first and second indicator values C1′ and C2′ are calculated from one or more pieces of biological information noninvasively measured for the experimental subject once. Further, in the biorhythm estimation unit 20, times t₁₂ and t₂₂ (t₁₁<t₁₂) corresponding to the first indicator value C1′ on the first rhythm curve EC1 are derived and times t₂₁ and t₂₂ (t21<t22) corresponding to the second indicator value C2′ on the second rhythm curve EC2 are derived. Also, the biorhythm of the experimental subject is estimated from a time corresponding to a smaller of a period d1 (=|t₁₁−t₂₁|) and a period d2 (=|t₁₂−t₂₂|).

Hereinafter, a case in which the first indicator value C1′ is a pulse rate of the experimental subject or the like and the second indicator value C2′ is an AI value of the experimental subject or the like will be described. However, a combination of the first indicator value C1′ and the second indicator value C2′ may be a combination of the pulse rate or the AI value and an internal temperature of an ear or a mouth or may be a combination of other indicator values. Hereinafter, a biorhythm estimation procedure based on a 1-point method will be described with reference to FIG. 11.

As illustrated in FIG. 11, first, the first reference rhythm curve RC1 and the first experimental-subject rhythm curve EC1 are prepared for an average pulse rate (step S51). Similarly, the second reference rhythm curve RC2 and the second experimental-subject rhythm curve EC2 are prepared for an average AI value (step S52). Further, the process in steps S51 and S52 is performed similarly to the procedure described with reference to FIGS. 5, 7 and 9.

The pulse wave is then measured in a measurement period of time y seconds at any time t, and an average pulse rate C1′(t) and an average AI value C2′(t) at the time t are calculated in the procedure described with reference to FIG. 7 (step S53).

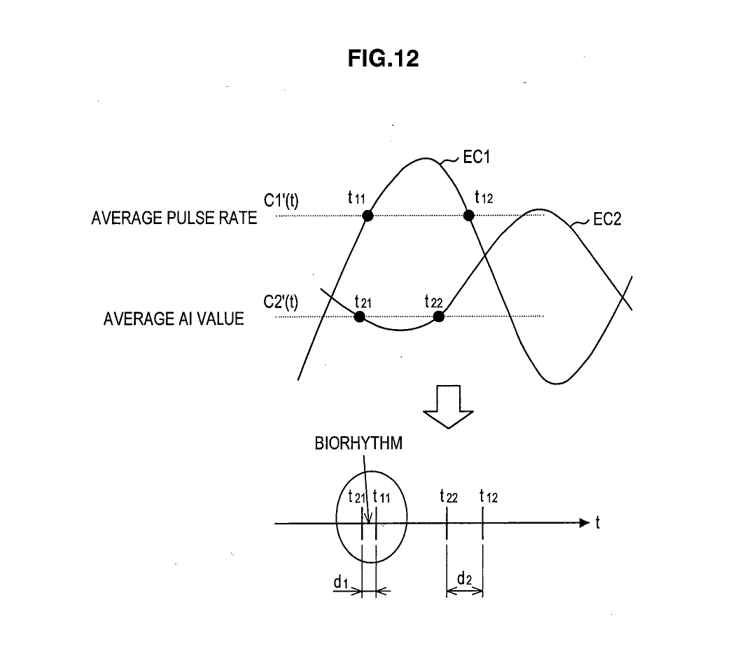

Next, as illustrated in FIG. 12, times t₁₁ and t₁₂ (t₁₁<t₁₂) corresponding to the average pulse rate C1′(t) are derived on the first experimental-subject rhythm curve EC1 representing a property of intra-day fluctuation of the pulse rate (step S54). Similarly, times t₂₁ and t₂₂ (t₂₁<t₂₂) corresponding to the average AI value C2′(t) are derived on the second experimental-subject rhythm curve EC2 representing a property of intra-day fluctuation of the average AI value (step S55).

An abnormal value is then rejected from the calculated times t₁₁, t₁₂, t₂₁, and t₂₂ (step S56). Here, for example, if a difference value between the time t₁₁ and the time t₂₁ or a difference value between the time t₂₁ and the time t₂₂ exceeds a predetermined threshold value, the abnormal value is rejected from the calculated times when the average pulse rate C1′(t) and the average AI value C2′(t) are both located above or both located below the baseline of the rhythm curve. When the abnormal value is rejected, the experimental subject is requested to perform re-measurement of the pulse wave.

Next, as illustrated in FIG. 12, a period d1=|t₁₁-t₂₁| is calculated from the times t₁₁ and t₂₁ and a period d2=|t₁₂-t₂₂| is calculated from the times t₁₂ and t₂₂ (step S57). Also, the smaller of the two periods d1 and d2 is selected (step S58) and, for example, a time in the middle of the time t₁₁ and the time t₂₁ (=t₁₁+(t₂₁₋t₁₁)/2) in the selected period is estimated as the biorhythm of the experimental subject (step S59).

(Biorhythm Estimation Based on 2-point Method)

In the 2-point method, a reference rhythm curve RC and an experimental-subject rhythm curve EC representing a property of intra-day fluctuation of the indicator value C′ are used. In the indicator value calculation unit 15, two indicator values C′(t₁) and C′(t₂) having different measurement time points are calculated from the biological information noninvasively measured for the experimental subject twice at a measurement interval Δt within 12 hours.

Also, in the biorhythm estimation unit 20, one or more times t₁₁ corresponding to the indicator value C′(t₁) are derived on the experimental-subject rhythm curve EC and one or more times t₂₁ and t₂₂ corresponding to the indicator value C′(t₂) are derived on the experimental-subject rhythm curve EC. Also, the biorhythm of the experimental subject is estimated from a time corresponding to the closest period to the measurement interval Δt among periods d between the time t₁₁ and the times t₂₁ and t₂₂.

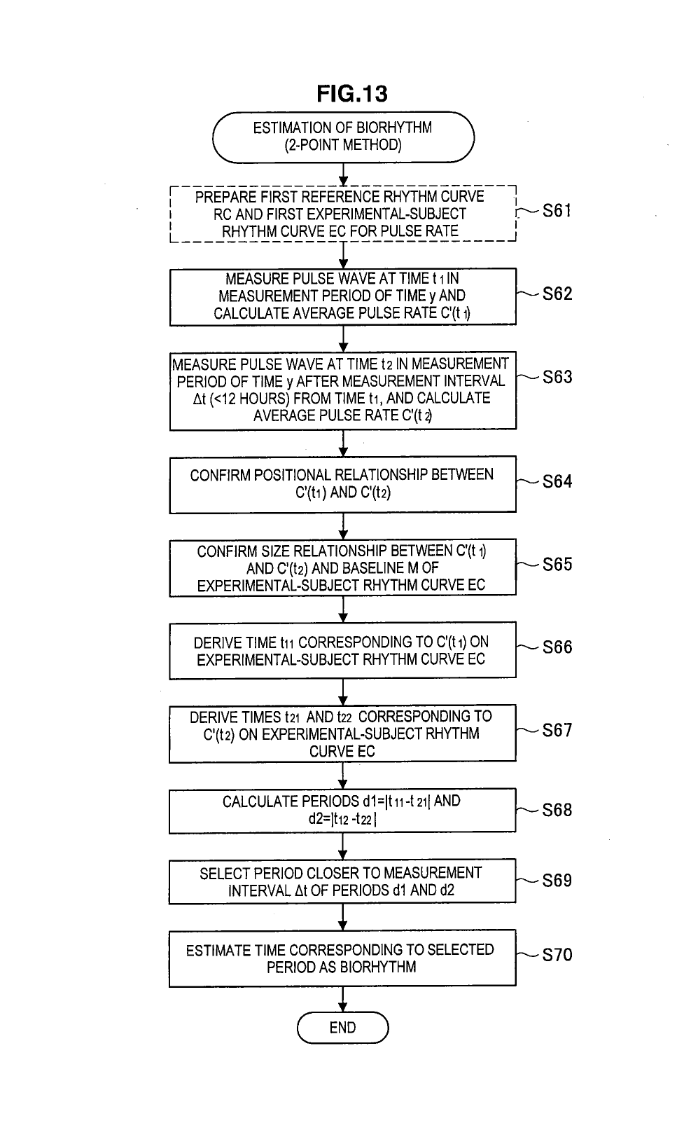

Hereinafter, a case in which the indicator value C′ is the pulse rate of the experimental subject or the like will be described. However, the indicator value C′ may be an AI value, an internal temperature of the ear or the mouth, or the like or may be another indicator value. Hereinafter, a biorhythm estimation procedure based on a 2-point method will be described with reference to FIG. 13.

As illustrated in FIG. 13, first, the reference rhythm curve RC and the experimental-subject rhythm curve EC are prepared for an average pulse rate (step S61). Further, a process of step S61 is performed similarly to the procedure described with reference to FIGS. 5, 7 and 9.

First, the pulse wave is measured in a measurement period of time y seconds at any time t, and an indicator value C′(t₁) at a time t=t₁ is calculated in the procedure described with reference to FIG. 7 (step S62). Similarly, the pulse wave is measured in the measurement period of time y seconds at a time t₂ after the measurement interval Δt (Δt<12 hours) from the time t₁, and the indicator value C′(t₂) at a time t₂ is calculated (step S63). Hereinafter, a case in which the measurement interval Δt is equal to 3 hours will be described, but the measurement interval Δt may not be 3 hours as long as the measurement interval is within 12 hours.

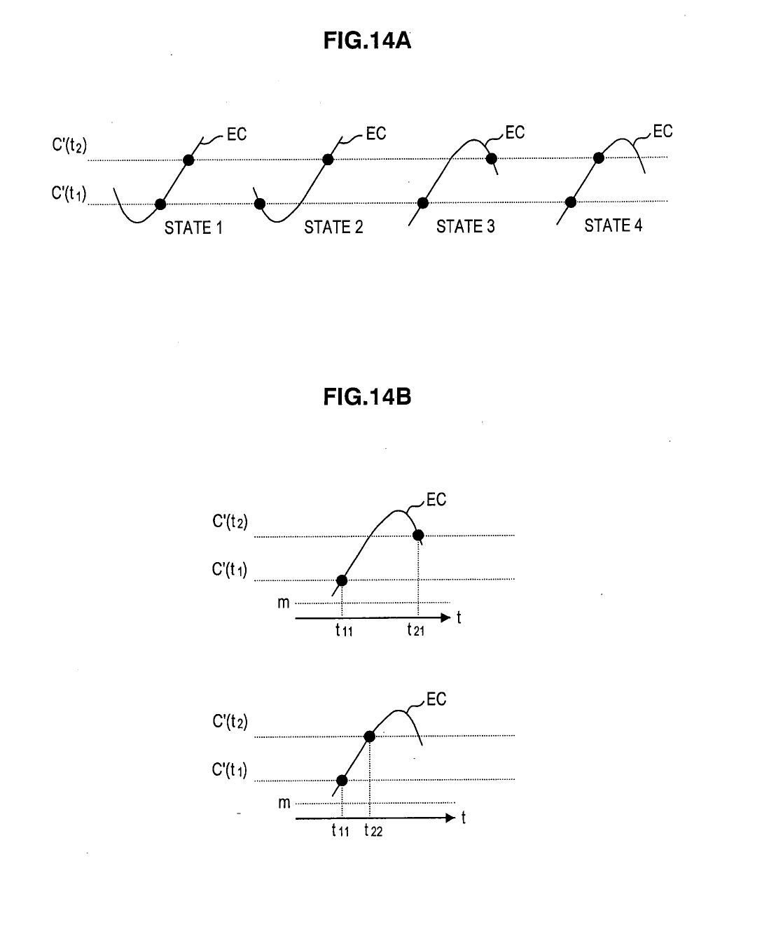

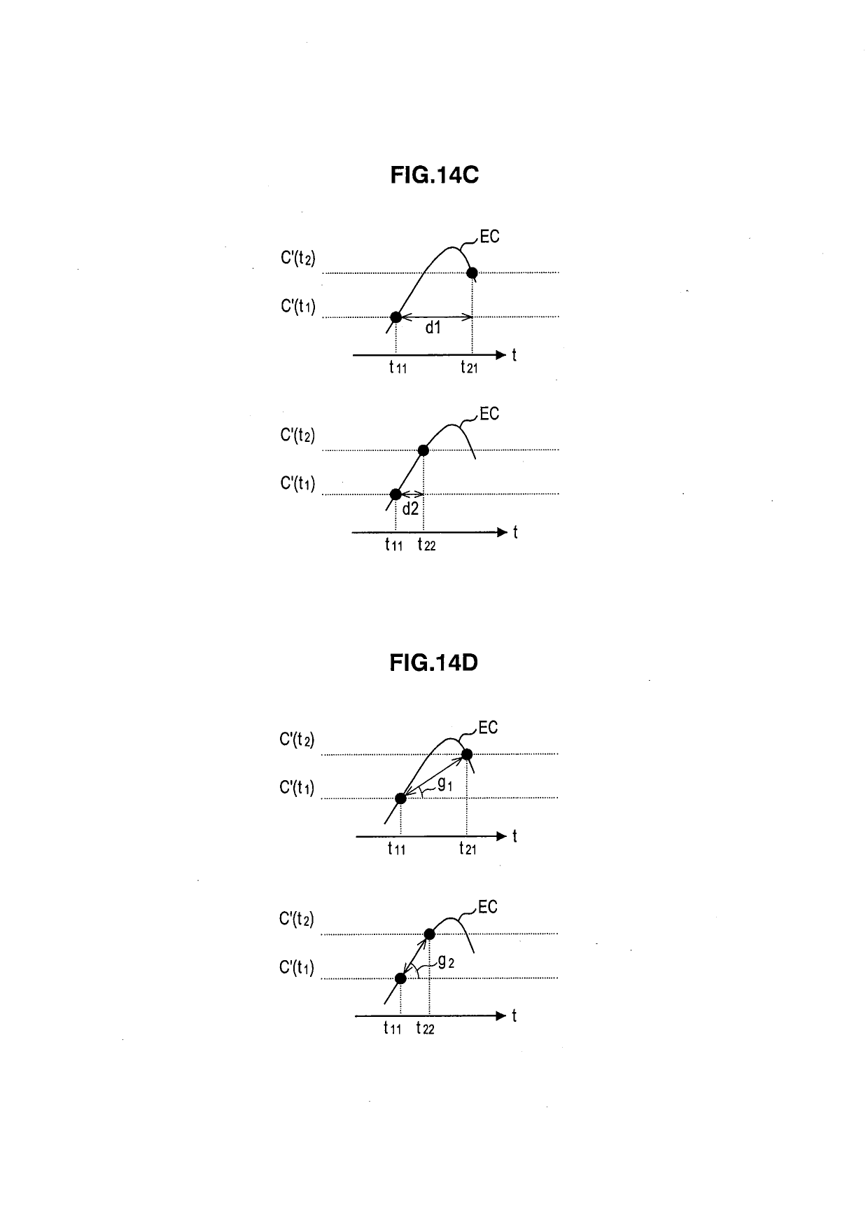

A positional relationship between the indicator values C′(t₁) and C′(t₂) is then estimated on the experimental-subject rhythm curve EC (step S64). As illustrated in FIG. 14A, the positional relationship between the indicator values C′(t₁) and C′(t₂) is estimated as any of the following four states.

state 1: C′(t1) and C′(t2) are not located across a lower convex portion of the curve EC or a peak of the curve EC.

state 2: C′(t1) and C′(t2) are located across a lower convex portion of the curve EC and a peak of the curve EC.

state 3: C′(t1) and C′(t2) are not located across an upper convex portion of the curve EC or a peak of the curve EC.

state 4: C′(t1) and C′(t2) are located across an upper convex portion of the curve EC and a peak of the curve EC.

A size relationship between the indicator values C′(t1) and C′(t2) and the baseline m of the curve EC is then confirmed (step S65). Accordingly, a determination is made as to whether the indicator values C′(t₁) and C′(t₂) are located in the upper convex portion or the lower convex portion of the curve EC. For example, when the indicator values C′(t₁) and C′(t₂) are greater than the baseline m as illustrated in FIG. 14B, state 3 or 4 is estimated as the positional relationship between the indicator values C′(t₁) and C′(t₂).

Next, as illustrated in FIG. 14C, times t₁₁, t₂₁, and t₂₂ corresponding to the indicator values C′(t1) and C′(t2) are calculated on the curve EC for states 3 and 4 (steps S66 and S67), and a period dl between a time t₁₁ and a time t₂₁ and a period d2 between the time t₁₁ and a time t₂₂ are calculated (step S68). Also, a period approximating the actual measurement interval Δt is selected of the calculated periods d1 and d2 (step S69). Also, the time t₁₁, t₂₁ or t₂₂ corresponding to the selected period is estimated as the biorhythm of the experimental subject (step S70).

Further, as illustrated in FIG. 14D, for states 3 and 4, a first gradient g1 may be calculated from the times t₁₁ and t₂₁ and the indicator values C′(t₁) and C′(t₂) and a second gradient g2 may be calculated from the times t₁₁ and t₂₂ and the indicator values C′(t₁) and C′(t₂). Also, a period with a gradient closer to the gradient calculated from a relationship between the actual measurement period Δt and the indicator values C′(t₁) and C′(t₂) is selected of the calculated gradients g1 and g2. Also, the time t₁₁, t₂₁ or t₂₂ corresponding to the selected gradient is estimated as the biorhythm of the experimental subject.

In the 2-point method, the 1-point method described above may be performed twice within 12 hours. Accordingly, when the first and second indicator values C1′(t) and C2′(t) can be calculated, the 2-point method may be applied to the first and second indicator values C1′(t) and C2′(t) to calculate the first and second estimation values. Also, when an error between the first and second estimation values is small, any one of the estimation values may be adopted or an average value of the two estimation values may be adopted. On the other hand, when the error between the first and second estimation values is great, the estimation is determined to fail and the experimental subject may be requested to perform re-measurement. Further, when one of the first and second indicator values C1′(t) and C2′(t) cannot be calculated for any reason, the 2-point method may be applied to the calculated indicator value to estimate the biorhythm.

(Biorhythm Estimation Based on Domain Method)

In the domain method, a reference rhythm curve RC and an experimental-subject rhythm curve EC representing a property of intra-day fluctuation of an indicator value are used. In the indicator value calculation unit 15, three or more indicator values C′(t1), C′(t2), C′(t₃), . . . with different measurement time points are calculated from the biological information noninvasively measured for the experimental subject three times or more.

Also, in the biorhythm estimation unit 20, three or more indicator values C′(t1), C′(t2), C′(t₃), . . . are applied to the experimental-subject rhythm curve EC to estimate the biorhythm of the experimental subject from times corresponding to the three or more indicator values on the experimental-subject rhythm curve EC.

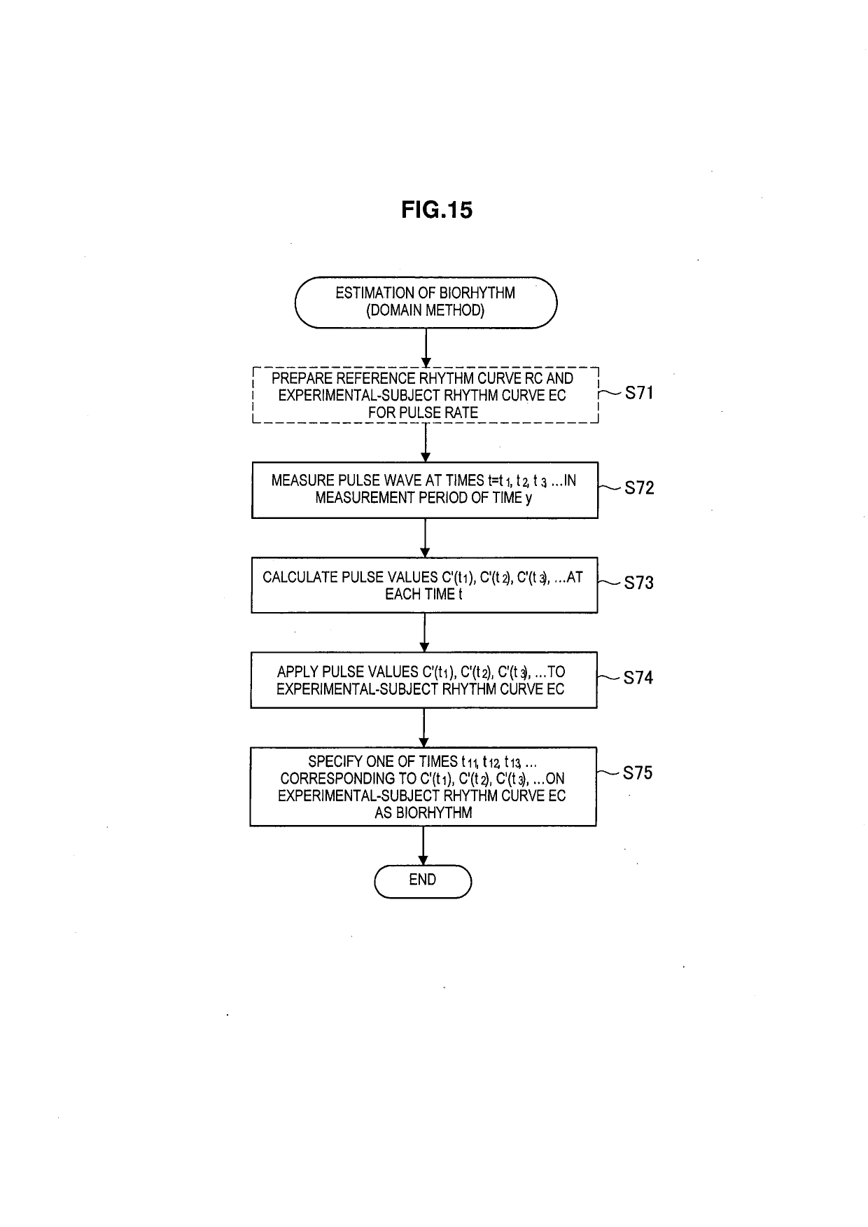

Hereinafter, a case in which the indicator value C′ is the pulse rate of the experimental subject or the like will be described. However, the indicator value C′ may be an AI value, an internal temperature of the ear or mouth, or the like or may be another indicator value. Hereinafter, a biorhythm estimation procedure based on the domain method will be described with reference to FIG. 15.

As illustrated in FIG. 15, first, a reference rhythm curve RC and an experimental-subject rhythm curve EC are prepared for an average pulse rate (step S71). Further, a process of step S71 is performed similarly to the procedure described with reference to FIGS. 5, 7 and 9.

A pulse wave is then measured in a measurement period of time y seconds at any three or more times t=t₁, t₂, t₃, . . . (step S72), and indicator values C′(t1), C′(t2), C′(t₃), . . . at a time t which is t₁, t₂, t₃, . . . are calculated in the procedure described with reference to FIG. 7 (step S73).

Here, the pulse wave may be measured as a continuous measurement value during sleep or while awake. In this case, the measured pulse wave is separated at certain time intervals such as 0.5 hours, 1 hour or the like, and indicator values C′(t1), C′(t2), C′(t₃), . . . are calculated in each section in the procedure described with reference to FIG. 7. Further, when the pulse wave is separated at certain time intervals, adjacent sections may partially overlap.

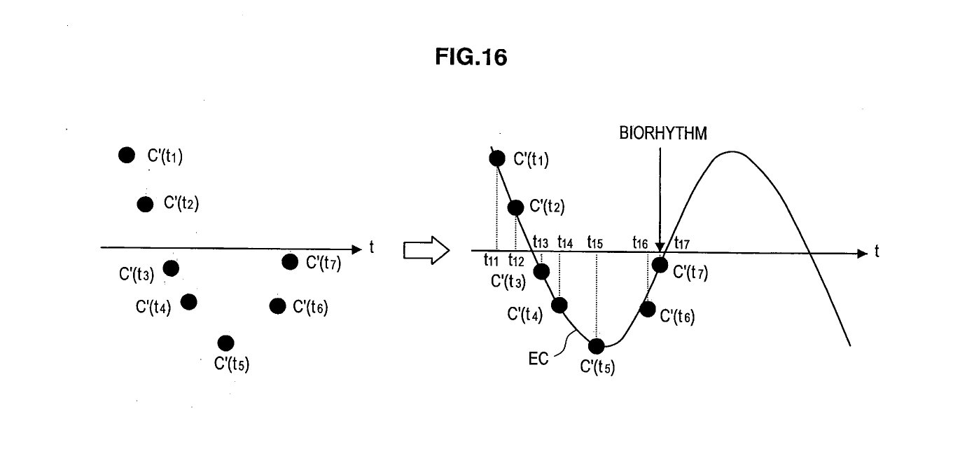

Next, as illustrated in FIG. 16, a series of indicator values C′(t1), C′(t2), C′(t₃) . . . are applied (curve-fitted) to the experimental-subject rhythm curve EC (step S74). In the curve fitting, the experimental-subject rhythm curve EC and a series of indicator values C′ may be shifted in a time direction to obtain a time t minimizing a difference value between the experimental-subject rhythm curve EC and the indicator value and to obtain a time t maximizing a correlation coefficient between the experimental-subject rhythm curve EC and the indicator value.

Also, at least one of the times t₁₁, t₁₂, t₁₃, . . . corresponding to the indicator values C′(t1), C′(t2), C′(t₃), . . . on the experimental-subject rhythm curve EC is estimated as the biorhythm of the experimental subject (step S75). For example, in an example illustrated in FIG. 16, a measurement value during sleep is used, and a time t₁₇ corresponding to an indicator value C′(t₇) upon waking up is estimated as the biorhythm.

Even in the domain method, the 1-point method described above may be performed three times or more. Accordingly, when the first and second indicator values C1′(t) and C2′(t) can be calculated, the domain method can be applied to the first and second indicator values C1′(t) and C2′(t) to calculate the first and second estimation values. Also, when the error between the first and second estimation values is small, any one of the estimation values may be adopted or an average value of two estimation values may be adopted. On the other hand, when the error between the first and second estimation values is great, the estimation is determined to have failed and the experimental subject may be requested to perform re-measurement. Further, even when one of the first and second indicator values C1′(t) and C2′(t) cannot be calculated for any reason, the domain method may be applied to the calculated indicator value to estimate the biorhythm.

### 4. CONCLUSION

As described above, according to the biorhythm estimation method in the embodiment of the present disclosure, the biological information which can be measured noninvasively and in a short time and from which the biorhythm is easily recognized, in other words, which has a goodness of fit of the biorhythm curve and the indicator value, is used in the estimation of the biorhythm.

Accordingly, the experimental subject does not experience pain, discomfort or the like due to invasive measurement and does not suffer in daily life due to long-period measurement, unlike a biorhythm estimation method of the related art. Thus, it is possible to estimate the biorhythm of the experimental subject based on the noninvasively and easily measured biological information.

The preferred embodiments of the present disclosure have been described above with reference to the accompanying drawings, whilst the present invention is not limited to the above examples, of course. A person skilled in the art may find various alternations and modifications within the scope of the appended claims, and it should be understood that they will naturally come under the technical scope of the present invention.

For example, in the above description, the case in which the pulse wave is used as the biological information has been mainly described. However, the biological information is not limited to the pulse wave, and other biological information which can be measured noninvasively and in a short time and from which the biorhythm is easily recognized, in other words, which has a goodness of fit of the biorhythm curve and the indicator value may be used.

### REFERENCE SIGNS LIST

1 biorhythm estimation device
11 biological information measurement sensor
12 disturbance information measurement sensor
13 measurement timer
14 measurement value storage unit
15 indicator value calculation unit
16 reference rhythm curve creation unit
17 reference rhythm curve storage unit
18 experimental-subject rhythm curve creation unit
19 experimental-subject rhythm curve storage unit
20 biorhythm estimation unit
21 biorhythm output unit
RC reference rhythm curve
EC experimental-subject rhythm curve

1. A biorhythm estimation device comprising:
a reference rhythm curve storage unit configured to store a reference rhythm curve based on a plurality of experimental subject samples, the reference rhythm curve being created as a biorhythm curve representing a property of intra-day fluctuation of an indicator value of biological information;
a biological information measurement unit configured to noninvasively measure the biological information of an experimental subject;
an indicator value calculation unit configured to calculate the indicator value from a measurement value of the biological information;
an experimental-subject rhythm curve creation unit configured to fit the reference rhythm curve to the indicator value calculated from the measurement value obtained by noninvasively measuring the biological information of the experimental subject to create an experimental-subject rhythm curve whose target is the experimental subject;
an experimental-subject rhythm curve storage unit configured to store the experimental-subject rhythm curve; and
a biorhythm estimation unit configured to apply the indicator value calculated from the measurement value obtained by noninvasively measuring the biological information of the experimental subject to the experimental-subject rhythm curve to estimate biorhythm of the experimental subject.

2. The biorhythm estimation device according to 1, wherein:
the reference rhythm curve is created from the indicator value calculated from the measurement value of the biological information measured for the plurality of experimental subject samples for 24 hours or more,
the experimental-subject rhythm curve creation unit fits the reference rhythm curve to two or more indicator values calculated from the biological information noninvasively measured for the experimental subject twice or more at a time interval, and
the biorhythm estimation unit applies the two or more indicator values calculated from the biological information noninvasively measured for the experimental subject once or more to the experimental-subject rhythm curve.

3. The biorhythm estimation device according to claim 2, wherein:
the reference rhythm curve and the experimental-subject rhythm curve have a first rhythm curve representing a property of intra-day fluctuation of a first indicator value calculated from one or more pieces of biological information and a second rhythm curve representing a property of intra-day fluctuation of a second indicator value whose type is different from the first indicator value, respectively,
the indicator value calculation unit calculates the first indicator value and the second indicator value from the one or more pieces of biological information noninvasively measured for the experimental subject once, and
the biorhythm estimation unit derives times t₁₁ and t₁₂ (t₁₁<t₁₂) corresponding the first indicator value on the first rhythm curve, derives times t₂₁ and t₂₂ (t₂₁<t₂₂) corresponding to the second indicator value on the second rhythm curve, and estimates the biorhythm of the experimental subject from a time corresponding to the smaller of a period |t₁₁-t₂₁| and a period |t₁₂-t₂₂|.

4. The biorhythm estimation device according to claim 3, wherein the first indicator value is a pulse rate calculated from a pulse wave, and the second indicator value is an AI value calculated from the pulse wave.

5. The biorhythm estimation device according to claim 3, wherein the first indicator value is a pulse rate or an AI value calculated from a pulse wave, and the second indicator value is an internal temperature of a mouth or an internal temperature of an ear.

6. The biorhythm estimation device according to claim 2, wherein:
the reference rhythm curve and the experimental-subject rhythm curve have a rhythm curve representing a property of intra-day fluctuation of the indicator value calculated from the biological information, respectively,
the indicator value calculation unit calculates a first indicator value and a second indicator value with different measurement time points from the biological information noninvasively measured for the experimental subject twice at a measurement interval within 12 hours, and
the biorhythm estimation unit derives one or more first times corresponding to the first indicator value on the experimental-subject rhythm curve, derives one or more second times corresponding to the second indicator value on the experimental-subject rhythm curve, and estimates the biorhythm of the experimental subject from the time corresponding to the closest period to the measurement interval among periods between the first times and the second times.

7. The biorhythm estimation device according to claim 6, wherein each of the first and second indicator values is a pulse rate or an AI value calculated from a pulse wave, or an internal temperature of a mouth or an internal temperature of an ear.

8. The biorhythm estimation device according to claim 2, wherein:
the reference rhythm curve and the experimental-subject rhythm curve have a rhythm curve representing a property of intra-day fluctuation of the indicator value calculated from the biological information, respectively,
the indicator value calculation unit calculates three or more indicator values with different measurement time points from the biological information noninvasively measured for the experimental subject three times or more at a time interval, and
the biorhythm estimation unit applies the three or more indicator values to the experimental-subject rhythm curve to estimate the biorhythm of the experimental subject from times corresponding to the three or more indicator values on the experimental-subject rhythm curve.

9. The biorhythm estimation device according to claim 8, wherein the indicator value is a pulse rate or an AI value calculated from a pulse wave, or an internal temperature of a mouth or an internal temperature of an ear.

10. The biorhythm estimation device according to claim 1, wherein:
the reference rhythm curve is prepared as a first rhythm curve whose target is a plurality of experimental subject samples having at least a first living-hour pattern, and a second rhythm curve whose target is a plurality of experimental subject samples having a second living-hour pattern different from the first living-hour pattern, and
the experimental-subject rhythm curve creation unit fits at least one of the first or second rhythm curve that matches a living-hour pattern of the experimental subject to the indicator value to create the experimental-subject rhythm curve.

11. The biorhythm estimation device according to claim 1, wherein the biorhythm curve is a single-mode function.

12. The biorhythm estimation device according to claim 11, wherein the single-mode function is a trigonometric function of a cycle of 24 hours.

13. A biorhythm estimation method comprising:
preparing a reference rhythm curve based on a plurality of experimental subject samples as a biorhythm curve representing a property of intra-day fluctuation of an indicator value of the biological information;
fitting the reference rhythm curve to the indicator value calculated from a measurement value obtained by noninvasively measuring the biological information of an experimental subject to create an experimental-subject rhythm curve whose target is the experimental subject; and
applying the indicator value calculated from the measurement value obtained by noninvasively measuring the biological information of the experimental subject to the experimental-subject rhythm curve to estimate biorhythm of the experimental subject.

14. A program for causing a computer to execute:
preparing a reference rhythm curve based on a plurality of experimental subject samples as a biorhythm curve representing a property of intra-day fluctuation of an indicator value of biological information;
fitting the reference rhythm curve to the indicator value calculated from a measurement value obtained by noninvasively measuring the biological information of an experimental subject to create an experimental-subject rhythm curve whose target is the experimental subject; and
applying the indicator value calculated from the measurement value obtained by noninvasively measuring the biological information of the experimental subject to the experimental-subject rhythm curve to estimate biorhythm of the experimental subject.
